SEQUENCE LISTING (45 sequences)

SEQ ID NO: 1 - protein, 39 aa, Heloderma suspectum
  C-term amidated
HGEGTFTSDLSKQMEEEAVRLFIEWLKNGGPSSGAPPPS

SEQ ID NO: 2 - protein, 39 aa, Heloderma horridum
  C-term amidated
HSDGTFTSDLSKQMEEEAVRLFIEWLKNGGPSSGAPPPS

SEQ ID NO: 3 - protein, 39 aa, Artificial Sequence
  Description of Artificial Sequence Synthetic polypeptide
HGEGTFTSDLSKQLEEEAVRLFIEWLKNGGPSSGAPPPS

SEQ ID NO: 4 - protein, 39 aa, Artificial Sequence
  Description of Artificial Sequence Synthetic polypeptide
HGEGTFTSDLSKQLEEEAVRLFIEFLKNGGPSSGAPPPS

SEQ ID NO: 5 - protein, 39 aa, Artificial Sequence
  Description of Artificial Sequence Synthetic polypeptide
HGEGTFTSDLSKQLEEEAARLFIEFLKNGGPSSGAPPPS

SEQ ID NO: 6 - protein, 30 aa, Artificial Sequence
  Description of Artificial Sequence Synthetic polypeptide
HGEGTFTSDLSKQMEEEAVRLFIEWLKNGG

SEQ ID NO: 7 - protein, 30 aa, Artificial Sequence
  Description of Artificial Sequence Synthetic polypeptide
HGEGTFTSDLSKQLEEEAVRLFIEWLKNGG

SEQ ID NO: 8 - protein, 30 aa, Artificial Sequence
  Description of Artificial Sequence Synthetic polypeptide
HGEGTFTSDLSKQLEEEAVRLFIEFLKNGG

SEQ ID NO: 9 - protein, 30 aa, Artificial Sequence
  Description of Artificial Sequence Synthetic polypeptide
HGEGTFTSDLSKQLEEEAARLFIEFLKNGG

SEQ ID NO: 10 - protein, 28 aa, Artificial Sequence
  Description of Artificial Sequence Synthetic peptide
HGEGTFTSDLSKQMEEEAVRLFIEWLKN

SEQ ID NO: 11 - protein, 28 aa, Artificial Sequence
  Description of Artificial Sequence Synthetic peptide
HGEGTFTSDLSKQLEEEAVRLFIEWLKN

SEQ ID NO: 12 - protein, 28 aa, Artificial Sequence
  Description of Artificial Sequence Synthetic peptide
HGEGTFTSDLSKQLEEEAVRLFIEFLKN

SEQ ID NO: 13 - protein, 28 aa, Artificial Sequence
  Description of Artificial Sequence Synthetic peptide
HGEGTFTSDLSKQLEEEAARLFIEFLKN

SEQ ID NO: 14 - protein, 39 aa, Artificial Sequence
  Description of Artificial Sequence Synthetic polypeptide
HGEGTFTSDLSKQLEEKAAKEFIEFLKQGGPSSGAPPPS

SEQ ID NO: 15 - protein, 39 aa, Artificial Sequence
  Description of Artificial Sequence Synthetic polypeptide
HGEGTFTSDLSKQLEEKAAKEFIEWLKQGGPSSGAPPPS

SEQ ID NO: 16 - protein, 39 aa, Artificial Sequence
  Description of Artificial Sequence Synthetic polypeptide
HGEGTFTSDLSKQGEEEAVRLFIEWLKQGGPSSGAPPPS

SEQ ID NO: 17 - protein, 39 aa, Artificial Sequence
  Description of Artificial Sequence Synthetic polypeptide
HGEGTFTSDLSKQLEEEAVRLFIEWLKQGGPSSGAPPPS

SEQ ID NO: 18 - protein, 37 aa, Artificial Sequence
  Description of Artificial Sequence Synthetic polypeptide
HGEFTFTSDLSKQLEEEAVRLFIEWLKQGGPSKEIIS

SEQ ID NO: 19 - protein, 39 aa, Artificial Sequence
  Description of Artificial Sequence Synthetic polypeptide
HGEFTFTSDLSKQLEEKAAKEFIEWLKQGGPSSGAPPPS

SEQ ID NO: 20 - protein, 39 aa, Artificial Sequence
  Description of Artificial Sequence Synthetic polypeptide
HGEGTFTSDLVKILEAEAVRKFIEFLKNGGPSSGAPPPS

SEQ ID NO: 21 - protein, 40 aa, Artificial Sequence
  Description of Artificial Sequence Synthetic polypeptide
HGEGTFTSDLSKQMEEEAVRLFIEWLKNGGPSSGAPPPSK

SEQ ID NO: 22 - protein, 31 aa, Homo sapiens
  C-term may or may not be amidated
HAEGTFTSDVSSYLEGQAAKEFIAWLVKGRG

SEQ ID NO: 23 - protein, 30 aa, Artificial Sequence
  Description of Artificial Sequence Synthetic polypeptide
HAEGTFTSDVSSYLEGQAAKEFIAWLVKGR

SEQ ID NO: 24 - protein, 30 aa, Artificial Sequence
  Description of Artificial Sequence Synthetic polypeptide
HXEGTFTSDVSSYLEGQAAREFIAFLVRXR

SEQ ID NO: 25 - protein, 31 aa, Artificial Sequence
  Description of Artificial Sequence Synthetic polypeptide
HXEGTFTSDVSSYLEXXAAKEFIXWLXXGXX

SEQ ID NO: 26 - protein, 31 aa, Artificial Sequence
  Description of Artificial Sequence Synthetic polypeptide
HAEGTFTSDVSSYLEGQAAKEFIAWLVRGRG

SEQ ID NO: 27 - protein, 31 aa, Artificial Sequence
  Description of Artificial Sequence Synthetic polypeptide
HAEGTFTSDVSSYLEGQAAKEFIEWLVKGRG

SEQ ID NO: 28 - protein, 31 aa, Artificial Sequence
  Description of Artificial Sequence Synthetic polypeptide
HAEGTFTSDVSSYLEKQAAKEFIAWLVKGRG

SEQ ID NO: 29 - protein, 31 aa, Artificial Sequence
  Description of Artificial Sequence Synthetic polypeptide
HGEGTFTSDVSSYLEEQAAKEFIAWLVKGGG

SEQ ID NO: 30 - protein, 31 aa, Artificial Sequence
  Description of Artificial Sequence Synthetic polypeptide
HVEGTFTSDVSSYLEEQAAKEFIAWLVKGGG

SEQ ID NO: 31 - protein, 31 aa, Artificial Sequence
  Description of Artificial Sequence Synthetic polypeptide
HGEGTFTSDVSSYLEEQAAKEFIAWLKNGGG

SEQ ID NO: 32 - protein, 31 aa, Artificial Sequence
  Description of Artificial Sequence Synthetic polypeptide
HVEGTFTSDVSSYLEEQAAKEFIAWLKNGGG

SEQ ID NO: 33 - protein, 31 aa, Artificial Sequence
  Description of Artificial Sequence Synthetic polypeptide
HGEGTFTSDVSSYLEEQAAKEFIAWLVKGGP

SEQ ID NO: 34 - protein, 31 aa, Artificial Sequence
  Description of Artificial Sequence Synthetic polypeptide
HVEGTFTSDVSSYLEEQAAKEFIAWLVKGGP

SEQ ID NO: 35 - protein, 31 aa, Artificial Sequence
  Description of Artificial Sequence Synthetic polypeptide
HGEGTFTSDVSSYLEEQAAKEFIAWLKNGGP

SEQ ID NO: 36 - protein, 31 aa, Artificial Sequence
  Description of Artificial Sequence Synthetic polypeptide
HVEGTFTSDVSSYLEEQAAKEFIAWLKNGGP

SEQ ID NO: 37 - protein, 30 aa, Artificial Sequence
  Description of Artificial Sequence Synthetic polypeptide
HGEGTFTSDVSSYLEEQAAKEFIAWLVKGG

SEQ ID NO: 38 - protein, 30 aa, Artificial Sequence
  Description of Artificial Sequence Synthetic polypeptide
HVEGTFTSDVSSYLEEQAAKEFIAWLVKGG

SEQ ID NO: 39 - protein, 30 aa, Artificial Sequence
  Description of Artificial Sequence Synthetic polypeptide
HVEGTFTSDVSSYLEEQAAKEFIAWLVNGG

SEQ ID NO: 40 - protein, 30 aa, Artificial Sequence
  Description of Artificial Sequence Synthetic polypeptide
HGEGTFTSDVSSYLEEQAAKEFIAWLVNGG

SEQ ID NO: 41 - protein, 230 aa, Artificial Sequence
  Description of Artificial Sequence Synthetic polypeptide
AESKYGPPCPPCPAPXXXGGPSVFLFPPKPKDTLMISRTPEVTCVVVDVSQEDPEVQFNWYVDGVEVHNAKTKPREEQFXSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 42 - protein, 30 aa, Artificial Sequence
  Description of Artificial Sequence Synthetic polypeptide
GGGGSGGGGSGGGGSGGGGSGGGGSGGGGS

SEQ ID NO: 43 - protein, 275 aa, Artificial Sequence
  Description of Artificial Sequence Synthetic polypeptide
HGEGTFTSDVSSYLEEQAAKEFIAWLVKGGGGGGGSGGGGSGGGGSAESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 44 - protein, 40 aa, Artificial Sequence
  Description of Artificial Sequence Synthetic polypeptide
HXEGTFTSDVSSYLEXQAAKEFIAWLXKGGPSSGAPPPCC

SEQ ID NO: 45 - protein, 40 aa, Artificial Sequence
  Description of Artificial Sequence Synthetic polypeptide
HVEGTFTSDVSSYLEEQAAKEFIAWLIKGGPSSGAPPPCC

(19) United States
(12) Patent Application Publication
(10) Pub. No.: US 2016/0361390 A1
(43) Pub. Date: Dec. 15, 2016

(54) GLP-1 RECEPTOR AGONIST COMPOUNDS FOR OBSTRUCTIVE SLEEP APNEA

(71) Applicants: Amylin Pharmaceuticals, LLC, Wilmington, DE (US); ELI LILLY & Company, Indianapolis, IN (US)

(72) Inventor: Eve Van Cauter, San Diego, CA (US)

(73) Assignees: Amylin Pharmaceuticals, LLC, Wilmington, DE (US); ELI LILLY & COMPANY, Indianapolis, IN (US)

(21) Appl. No.: 15/248,016

(22) Filed: Aug. 26, 2016

Related U.S. Application Data
(63) Continuation of application No. 14/728,488, filed on Jun. 2, 2015.
Application No. 14/728,488 is a continuation of application No. 14/045,332, filed on Oct. 3, 2013, now abandoned.
Application No. 14/045,332 is a continuation of application No. 13/508,527, filed on Aug. 7, 2012, now abandoned.
(60) Provisional application No. 61/257,603, filed on Nov. 3, 2009.

(51) Int. Cl. A61K 38/26; A61K 47/48

(52) U.S. Cl. CPC A61K 38/26; A61K 47/48415; A61K 47/48369

(57) ABSTRACT

The disclosure provides, among other things, the use of GLP-1 receptor agonist compounds to treat obstructive sleep apnea. The GLP-1 receptor agonist compounds may be exendins, exendin analogs, GLP-1 (7-37), GLP-1 (7-37) analogs (e.g., GLP-1 (7-36)-NH₂) and the like. The GLP-1 receptor agonist compound may be exenatide.

### CROSS REFERENCE TO RELATED APPLICATIONS

This application is a continuation of U.S. application Ser. No. 14/728,488, filed Jun. 2, 2015, which is a continuation of U.S. application Ser. No. 14/045,332, filed Oct. 3, 2013, which is a continuation of U.S. application Ser. No. 13/508,527, filed Aug. 7, 2012, which is a National Stage Entry of International Application No. PCT/US2010/054553, filed Oct. 28, 2010, which claims priority from U.S. Provisional Application No. 61/257,603 filed Nov. 3, 2009; all of which are incorporated herein by reference in their entireties.

### FIELD

The description is directed to the field of medicine, more particularly, to sleep.

### BACKGROUND

Obstructive sleep apnea is caused by a blockage of the airway, which usually occurs when the soft tissue in the throat collapses and closes during sleep. The blockage can occur in a portion of the pharyngeal lumen and may include obstructions formed by the collapse of the tongue against the posterior wall of the pharynx, the collapse of the lateral pharyngeal walls, and the combined collapse of the tongue with impingement of the soft palate, particularly the posterior portion of the soft palate including the uvula. During each apnea event, the brain briefly arouses the sufferer in order to initiate the resumption of breathing. This type of sleep, however, is extremely fragmented and of poor quality.

According to the National Institutes of Health, obstructive sleep apnea affects more than twelve million Americans. Obstructive sleep apnea may result in high blood pressure, cardiovascular disease, weight gain, impotency, headaches, and memory problems if it is not treated. Despite the seriousness of obstructive sleep apnea, a general lack of awareness among the public and healthcare professionals results in the vast majority of obstructive sleep apnea sufferers remaining undiagnosed and untreated,

Many devices have been developed to treat obstructive sleep apnea; however, the devices can be uncomfortable for the patients. There exists a need in the art to identify therapeutic agents that can be used to enhance the quality of sleep and treat sleep apnea without causing patient discomfort. The present disclosure is directed to these, as well as other, important ends.

### SUMMARY

Provided herein are methods to treat obstructive sleep apnea in patients in need thereof by administering to the patients therapeutically effective amounts of GLP-1 receptor agonist compounds to treat obstructive sleep apnea. Methods for treating obstructive sleep apnea include methods for reducing the severity of obstructive sleep apnea.

In some embodiments, obstructive sleep apnea may be treated by administering BYETTA® (exenatide, Amylin Pharmaceuticals, Inc., San Diego, Calif., and Eli Lilly and Co., Indianapolis, Ind.) to the patient. BYETTA® is a pharmaceutical composition that generally comprises a GLP-1 receptor agonist compound (e.g., exenatide), a preservative (e.g.,metacresol), a tonicity-adjusting agent (e.g., mannitol), and a buffer (e.g., an acetate buffer).

In some embodiments, obstructive sleep apnea may be treated by administering BYDUREON™ or exenatide once weekly (EQW) (Amylin Pharmaceuticals, Inc., Eli Lilly and Company, Alkermes, Inc.) to the patient. Exenatide once weekly is a pharmaceutical composition that comprises biodegradable microspheres (e.g., poly(lactide-co-glycolide) microspheres) and exenatide. Exenatide once weekly is described, for example, in WO 2007/024700, the disclosure of which is incorporated by reference herein.

For the methods described herein, the patient may be a mammal, such as a human. Conditions such as obstructive sleep apnea are usually more common in diabetic and/or obese patients, such that the patient in these methods of treatment is more likely to be diabetic, obese, or diabetic and obese.

### DETAILED DESCRIPTION

The description provides methods of using GLP-1 receptor agonist compounds to treat obstructive sleep apnea in patients in need thereof. The GLP-1 receptor agonist compounds are administered to patients in therapeutically effective amounts. The patient may be a mammal, such as a human. The description provides methods of using pharmaceutical compositions comprising GLP-1 receptor agonist compounds to treat obstructive sleep apnea in patients in need thereof. The pharmaceutical compositions comprise therapeutically effective amounts of the GLP-1 receptor agonist compounds. The compositions may be immediate release (e.g., administered qid, bid, tid) or extended release (e.g., administered QW or once a month). The patient may be a mammal, such as a human.

In one embodiment, the description provides methods to treat obstructive sleep apnea by administering therapeutically effective amounts of GLP-1 receptor agonist compounds to patients in need thereof. A patient with obstructive sleep apnea stops breathing numerous times, for varying amounts of time, during sleep. Methods for treating obstructive sleep apnea include reducing the number of times a patient stops breathing during the night and/or reducing the length of time a patient stops breathing. “Reducing” includes eliminating the symptoms of obstructive sleep apnea.

Obstructive sleep apnea may be classified by the Apnea-Hypopnea Index (AHI) which measures the total number of episodes of apnea and hypopnea occurring during sleep divided by the hours of sleep time. The AHI classifies sleep apnea as normal (i.e., less than 5 pauses in breathing lasting more than 10 seconds); mild (i.e., 5-15 pauses in breathing lasting more than 10 seconds); moderate (i.e., 15-30 pauses in breathing lasting more than 10 second); or severe (i.e., more than 30 pauses in breathing lasting more than 10 seconds), The methods of treating sleep apnea described herein include reducing the severity of obstructive sleep apnea based on the AHI. In one embodiment, the methods described herein reduce the patient's AHI from severe to moderate; or from severe to mild; or from severe to normal. In one embodiment, the methods described herein reduce the patient's ART from moderate to mild, or from moderate to normal. In one embodiment, the methods described herein reduce the patient's AHI from mild to normal.

A “GLP-1 receptor agonist compound” refers to compounds having GLP-1 receptor activity. Such exemplary compounds include exendins, exendin analogs, exendin agonists, GLP-1(7-37), GLP-1(7-37) analogs, GLP-1(7-37) agonists, and the like.

The term “exendin” includes naturally occurring (or synthetic versions of naturally occurring) exendin peptides that are found in the salivary secretions of the Gila monster. Exendins of particular interest include exendin-3 and exendin-4. The exendins, exendin analogs, and exendin agonists for use in the methods described herein may optionally be amidated, and may also be in an acid form, pharmaceutically acceptable salt form, or any other physiologically active form of the molecule.

Exendin-4 (HGEGTFTSDLSKQMEEEAVRLFIEWLKNGGPSSGAPPPS-NH ₂ (SEQ ID NO:1)) is a peptide found in the saliva of the Gila monster, Heloderma suspectum; and exendin-3 (HSDGTFTSDLSKQMEEEAVRLF1EWLKNGG PSSGAPPPS-NH₂, (SEQ ID NO:2)) is a peptide found in the saliva of the beaded lizard, Heloderma horridum. Exendins have some amino acid sequence similarity to some members of the glucagon-like peptide (GLP) family. For example, exendin-4 has about 53% sequence identity with glucagon-like peptide-1(GLP-1)(7-37) (HAEGTFTSDVSSYLEGQAAKEFIAWLVKGRG (SEQ ID NO:22)). However, exendin-4 is transcribed from a distinct gene, not the Gila monster homolog of the mammalian proglucagon gene from which GLP-1 is expressed. Additionally, exendin-4 is not an analog of GLP-1(7-37) because the structure of synthetic exendin-4 peptide was not created by sequential modification of the structure of GLP-1. Nielsen et al, Current Opinion in Investigational Drugs, 4(4):401-405 (2003).

Synthetic exendin-4, also known as exenatide, is commercially available as BYETTA® (Amylin Pharmaceuticals, Inc. and Eli Lilly and Company). BYETTA® contains exenatide, a preservative (e.g., metacresol), a tonicity-adjusting agent (e.g., mannitol), and a buffer (e.g., an acetate buffer). A once weekly formulation of exenatide is currently in development and is described in WO 2005/102293, the disclosure of which is incorporated by reference herein. This once weekly formulation comprises exenatide and biodegradable polymeric (e.g., poly(lactide-co-glycolide)) microspheres, and is referred to herein as EQW or BYDUREON™ (Amylin Pharmaceuticals, Inc., Eli Lilly and Company, Alkermes, Inc.).

“Exendin analog” refers to peptides or other compounds which elicit a biological activity of an exendin reference peptide, preferably having a potency equal to or better than the exendin reference peptide (e.g., exendin-4), or within five orders of magnitude (plus or minus) of potency compared to the exendin reference peptide, when evaluated by art-known measures such as receptor binding and/or competition studies as described, e.g., by Hargrove et al, Regulatory Peptides, 141:113-119 (2007), the disclosure of which is incorporated by reference herein. Preferably, the exendin analogs will bind in such assays with an affinity of less than 1 μM, and more preferably with an affinity of less than 3 nM, or less than 1 nM. The term “exendin analog” may also be referred to as “exendin agonist”.

Exendin analogs also include the peptides described herein which have been chemically derivatized or altered, for example, peptides with non-natural amino acid residues (e.g., taurine, (β-amino acid residues, γ-amino acid residues, and D-amino acid residues), C-terminal functional group modifications, such as amides, esters, and C-terminal ketone modifications and N-terminal functional group modifications, such as acylated amines, Schiff bases, or cyclization, as found, for example, in the amino acid pyroglutamic acid. Exendin analogs may also contain other chemical moieties, such as peptide mimetics.

In one embodiment, the term “exendin analog” refers to a peptide having at least 75% sequence identity to exendin-4. In other embodiments, the term “exendin analog” refers to peptides having at least 80% sequence identity to exendin-4; at least 85% sequence identity to exendin-4; at least 90% sequence identity to exendin-4; or at least 95% sequence identity to exendin-4.

Exemplary exendins and exendin analogs exendin-4 (SEQ ID NO:1); exendin-3 (SED ID NO:2); Leu¹⁴-exendin-4 (SEQ ID NO:3); Leu¹⁴,Phe²⁵-exendin-4 (SEQ ID NO:4); Leu¹⁴,Ala¹⁹,Phe²⁵-exendin-4 (SEQ ID NO:5); exendin-4(1-30) (SEQ ID NO:6); Leu¹⁴-exendin-4(1-30) (SEQ ID NO:7); Leu¹⁴,Phe²⁵-exendin-4(1-30) (SEQ ID NO:8); Leu¹⁴,Ala¹⁹,Phe²⁵-exendin-4(1-30) (SEQ ID NO:9); exendin-4(1-28) (SEQ ID NO:10); Leu¹⁴-exendin-4(1-28) (SEQ ID NO:11); Leu¹⁴,Phe²⁵-exendin-4(1-28) (SEQ ID NO:12); Leu¹⁴,Ala¹⁹,Phe²⁵-exendin-4(1-28) (SEQ ID NO:13); Leu¹⁴,Lys^{17,20},Ala¹⁹,Glu²¹,Phe²⁵,Gln²⁸-exendin-4 (SEQ ID NO:14); Leu¹⁴,Lys^{17,20},Ala¹⁹,Glu²¹,Gln²⁸-exendin-4 (SEQ ID NO:15); octyGlyl¹⁴,Gln²⁸-exendin-4 (SEQ ID NO:16); Leu¹⁴,Gln²⁸,octylGly³⁴-exendin-4 (SEQ ID NO:17); Phe⁴,Leu¹⁴,Gln²⁸,Lys³³,Glu³⁴, Ile^{35,36}Ser³⁷-exendin-4(1-37) (SEQ ID NO:18); Phe⁴,Leu¹⁴,Lys^{17,20},Ala¹⁹ Glu²¹, Gln²⁸-exendin-4(SEQ ID NO:19); Val¹¹,Ile¹³,Leu¹⁴,Ala¹⁶,Lys²¹,Phe²⁵-exendin-4 (SEQ ID NO:20); exendin-4-Lye⁴⁰ (SEQ ID NO:21); lixisenatide (Sanofi-Aventis/Zealand Pharma); CJC-1134 (ConjuChem, Inc.); [N^{ε}-(17-carboxyheptadecanoic acid)Lys²⁰]exendin-4-NH₂; [N^{ε}-(17-carboxyhepta-decanoyl)Lys³²]exendin-4-NH₂; [desamino-His¹,N^{ε}-(17-carboxyheptadecanoyl)Lys²⁰]exendin-4-NH₂; [Arg^{12,27}NLe¹⁴,N^{ε}-(17-carboxy-heptadecanoyl)Lys³²] exendin-4-NH₂; [N^{ε}-(19-carboxy-nonadecanoylamino)Lys²⁰]-exendin-4-NH₂; [N^{ε}-(15-carboxypentadecanoylamino)Lys²⁰]-exendin-4-NH₂; [N^{ε}-(13-carboxytridecanoylamino)Lys²⁰]exendin-4-NH₂; [N^{ε}-(11-carboxy-undecanoyl-amino)Lys²⁰]exendin-4-NH₂; exendin-4-Lys⁴⁰(ε-MPA)-NH₂; exendin-4-Lys⁴⁰(ε-AEEA-MPA)-NH₂; exendin-4-Lys⁴⁰(ε-AEEA-MPA)-NH₂; exendin-4-Lys⁴⁰(ε-MPA)-albumin; exendin-4-Lys⁴⁰(ε-AEEA-AEEA-MPA)-albumin; exendin-4-Lys⁴⁰(ε-AEEA-MPA)-albumin; and the like. AEEA refers to [2-(2-amino)ethoxy)]ethoxy acetic acid. EDA refers to ethylenediamine. MPA refers to maleimidopropionic acid. The exendins and exendin analogs may optionally be amidated.

Other exendins and exendin analogs useful in the methods described herein include those described in WO 98/05351; WO 99/07404; WO 99/25727; WO 99/25728; WO 99/40788; WO 00/41546; WO 00141548; WO 00/73331; WO 01/51078; WO 03/099314; U.S. Pat. No. 6,956,026; U.S. Pat. No. 6,506,724; U.S. Pat. No. 6,703,359; U.S. Pat. No. 6,858,576; U.S. Pat. No, 6,872,700; U.S. Pat. No. 6,902,744; U.S. Pat. No. 7,157,555; U.S. Pat. No. 7,223,725; U.S. Pat. No. 7,220,721; US Publication No. 2003/0036504; and US Publication No. 2006/0094652, the disclosures of which are incorporated by reference herein in their entirety.

“GLP-1(7-37) analogs” refers to peptides or other compounds which elicit a biological activity similar to that of GLP-1(7-37), when evaluated by art-known measures such as receptor binding assays or in vivo blood glucose assays as described, e.g., by Hargrove et al, Regulatory Peptides, 141:113-119 (2007), the disclosure of which is incorporated by reference herein. In one embodiment, the term “GLP-1(7-37) analog” refers to a peptide that has an amino acid sequence with 1, 2, 3, 4, 5, 6, 7 or 8 amino acid substitutions, insertions, deletions, or a combination of two or more thereof, when compared to the amino acid sequence of GLP-1(7-37). In one embodiment, the GLP-1(7-37) analog is GLP-1(7-36)-NH₂. GLP-1(7-37) analogs include the amidated forms, the acid form, the pharmaceutically acceptable salt form, and any other physiologically active form of the molecule.

In one embodiment, the term “GLP-1(7-37)” refers to a peptide having at least 75% sequence identity to GLP-1(7-37). In other embodiments, the term “GLP-1(7-37) analog” refers to peptides having at least 80% sequence identity to GLP-1(7-37); at least 85% sequence identity to GLP-1(7-37); at least 90% sequence identity to GLP-1(7-37); or at least 95% sequence identity to GLP-1(7-37).

Exemplary GLP-1(7-37) and GLP-1(7-37) analogs include GLP-1(7-37) (SEQ ID NO:22); GLP-1(7-36))-NH₂ (SEQ ID NO:23); liraglutide (VICTOZA® from Novo Nordisk); albiglutide (SYNCRIA® from GlaxoSmithKline); taspoglutide (Hoffman La-Roche); LY2189265 (Eli Lilly and Company); LY2428757 (Eli Lilly and Company); desamino-His⁷,Arg²⁶,Lys³⁴ (N^{ε}-(γ-Glu(N-α-hexadecanoyl)))-GLP-1(7-37); desamino-His⁷,Arg²⁶,Lys³⁴(N^{ε}-octanoyl)-GLP-1(7-37); Arg^{26,34}Lys³⁸(N^{ε}-(ω-carboxypentadecanoyl))-GLP-1(7-38); Arg^{26,34}Lys³⁶(N^{ε}-(γ-Glu(N-α-hexadecanoyl)))-GLP-1(7-36); Aib^{8,35},Arg^{26,34},Phe³¹-GLP-1(7-36)) (SEQ ID NO:24); HXaa₈EGTFTSDVSSYLEXaa₂₂Xaa₂₃AAKEFIXaa₃₀WLXaa₃₃Xaa₃₄GXaa₃₆Xaa₃₇; wherein Xaa₈ is A, V, or G; Xaa₂₂ is G, K, or E; Xaa₂₃ is Q or K; Xaa₃₀ is A or E; Xaa₃₃ is V or K; Xaa₃₄ is K, N, or R; Xaa₃₆ is R or G; and Xaa₃₇ is G, H, P, or absent (SEQ ID NO:25); Arg³⁴-GLP-1(7-37) (SEQ ID NO:26); Glu³⁰-GLP-1(7-37) (SEQ ID NO:27): Lys22-GLP-1(7-37) (SEQ ID NO:28); Gly^{8,36},Glu²²-GLP-1(7-37) (SEQ ID NO:29); Val⁸,Glu²²,Gly³⁶-GLP-1(7-37) (SEQ ID NO:30); Gly^{8,36},Glu²²,Lys³³,Asn³⁴-GLP-1 (7-37) (SEQ ID NO:31); Val⁸,Glu²²,Lys³³,Asn³⁴,Gly³⁶-GLP-1(7-37) (SEQ ID NO:32); Gly^{8,36},Glu²²,Pro³⁷-GLP-1(7-37) (SEQ ID NO:33); Val⁸,Glu²²,Gly³⁶Pro³⁷-GLP-1 (7-37) (SEQ ID NO:34); Gly^{8,36} Glu²²,Lys³³,Asn³⁴,Pro³⁷-GLP-1(7-37) (SEQ ID NO:35); Val⁸,Glu²²,Lys³³,Asn³⁴,Gly³⁶,Pro³⁷-GLP-1(7-37) (SEQ ID NO:36); Gly^{8,36},Glu²²-GLP-1(7-36) (SEQ ID NO:37); Val⁸,Glu²²,Gly³⁶-GLP-1(7-36) (SEQ ID NO:38); Val⁸,Glu²²,Asn³⁴, Gly³⁶-GLP-1(7-36) (SEQ ID NO:39); Gly^{8,36},Glu²²,Asn³⁴-GLP-1(7-36) (SEQ ID NO:40). Each of the GLP-1(7-37) and GLP-1(7-37) analogs may optionally be amidated,

In one embodiment, the GLP-1(7-37) or GLP-1(7-37) analogs are covalently linked (directly or by a linking group) to an Fc portion of an immunoglobulin (e.g., IgG, IgE, IgG, and the like). For example, any one of SEQ ID NOs:25-40 may be covalently linked to the Fc portion of an immunoglobulin comprising the sequence of: AESKYGPPCPPCPAPXaa₁₆ Xaa₁₇Xaa₁₈GGPSVFLFPPKPKDTLMISRTPEVTCVVVDVSQEDPEVQFNWYVDGVEVHNAKTKPREEQFXaa₈₀STYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSL SLGXaa₂₃₀; wherein Xaa₁₆ is P or E; Xaa₁₇ is F, V or A; Xaa₁₈ is L, E or A; Xaa₈₀ is N or A; and Xaa₂₃₀ is K or absent (SEQ ID NO:41). The linking group may be any chemical moiety (e.g., amino acids and/or chemical groups). In one embodiment, the linking group is (-GGGGS-)ₓ (SEQ ID NO:42) where x is 1, 2, 3, 4, 5 or 6; preferably 2, 3 or 4; more preferably 3. In one embodiment, the GLP-1(7-37) analog covalently linked to the Fc portion of an immunoglobulin comprises the amino acid sequence:

(SEQ ID NO: 43)
HGEGTFTSDVSSYLEEQAAKEFIAWLVKGGGGGGGSGGGGSGGGGSAESK
YGPPCPPCPAPEAAGGPSVFLFPPKPKDTLMISRTPEVTCVVVDVSQEDP
INQFNWYVDGVEVHNAKTKPREEQFNSTYRVVSVLTVLHQDWLNGKEYKC
KVSNKGLPSSIEKTISKAKGQPREPQVYTLPPSQEEMTKNQVSLTCLVKG
FYPSDIAVEWESNKGQPENNYKTTPPVLDSDGSFFLYSRLTVDKSRWQEG
NVESCSVMHEALHNHYTQKSLSLSLG.

In another embodiment, the GLP-1(7-37) or GLP-1(7-37) analog may be covalently linked (directly or through a linking group) to one or two polyethylene glycol molecules. For example, a GLP-1(7-37) analog may comprise the amino acid sequence: HXaa₈EGTFTSDVSSYLEXaa₂₂QAAKEFIAWLXaa₃₃KGGPSSGAPPPC₄₅C₄₆—Z, wherein Xaa₈ is: D-Ala, G, V, L, I, S or T; Xaa₂₂ is G, E, D or K; Xaa₃₃ is: V or I; and Z is OH or NH₂, (SEQ ID NO:44), and, optionally, wherein (i) one polyethylene glycol moiety is covalently attached to C₄₅ (ii) one polyethylene glycol moiety is covalently attached to C₄₆, or (iii) one polyethylene glycol moiety is attached to C₄₅ and one polyethylene glycol moiety is attached to C₄₆. In one embodiment, the GLP-1(7-37) analog is HVEGTFTSDVSSYLEEQAAKEFIAWLIKGGPSSCAPPPC₄₅C₄₆—NH₂, (SEQ ID NO:45) and, optionally, wherein (i) one polyethylene glycol moiety is covalently attached to C₄₅, (ii) one polyethylene glycol moiety is covalently attached to C₄₆, or (iii) one polyethylene glycol moiety is attached to C₄₅ and one polyethylene glycol moiety is attached to C₄₆.

GLP-1 receptor agonist compounds may be prepared by processes well known in the art, e.g., peptide purification as described in Eng et al, J Biol. Chem., 265:20259-62 (1990); standard solid-phase peptide synthesis techniques as described in Raufman et al, J. Biol. Chem., 267:21432-37 (1992); recombinant DNA techniques as described in Sambrook et al, Molecular Cloning: A Laboratory Manual, 2d Ed., Cold Spring Harbor (1989); and the like.

The disclosure also provides pharmaceutical compositions comprising the GLP-1 receptor agonist compounds described herein and a pharmaceutically acceptable carrier. The GLP-1 receptor agonist compounds can be present in the pharmaceutical composition in a therapeutically effective amount and can be present in an amount to provide a minimum blood plasma level of the GLP-1 receptor agonist compound necessary for therapeutic efficacy. Such pharmaceutical compositions are known in the art and described, e.g., in U.S. Pat. No. 7,521,423; U.S. Pat. No. 7,456,254; WO 2000/037098; WO 2005/021022; WO 2005/102293; WO 2006/068910; WO 2006/125763; WO 2009/068910; US Publication No 2004/0106547; and the like, the disclosures of which are incorporated herein by reference.

Pharmaceutical compositions containing the GLP-1 receptor agonist compounds described herein may be provided for peripheral administration, such as parenteral (e.g., subcutaneous, intravenous, intramuscular), a continuous infusion (e.g., intravenous drip, intravenous bolus, intravenous infusion), topical, nasal, or oral administration. Suitable pharmaceutically acceptable carriers and their formulation are described in standard formulation treatises, such as Remington's Pharmaceutical Sciences by Martin; and Wang et al, Journal of Parenteral Science and Technology, Technical Report No. 10, Supp. 42:2S (1988).

The GLP-1 receptor agonist compounds described herein can be provided in parenteral compositions for injection or infusion. They can, for example, be suspended in water; an inert oil, such as a vegetable oil (e.g., sesame, peanut, olive oil, and the like); or other pharmaceutically acceptable carrier. In one embodiment, the compounds are suspended in an aqueous carrier, for example, in an isotonic buffer solution at a pH of about 3.0 to 8.0, or about 3.0 to 5.0. The compositions may be sterilized by conventional sterilization techniques or may be sterile filtered. The compositions may contain pharmaceutically acceptable auxiliary substances as required to approximate physiological conditions, such as pH buffering agents. Useful buffers include for example, acetic acid buffers. A form of repository or “depot” slow release preparation may be used so that therapeutically effective amounts of the preparation are delivered into the bloodstream over many hours or days following subcutaneous injection, transdermal injection or other delivery method. The desired isotonicity may be accomplished using sodium chloride or other pharmaceutically acceptable agents such as dextrose, boric acid, sodium tartrate, propylene glycol, polyols (such as mannitol and sorbitol), or other inorganic or organic solutes. In one embodiment for intravenous infusion, the formulation may comprise (i) the GLP-1 receptor agonist compound, (2) sterile water, and, optionally (3) sodium chloride, dextrose, or a combination thereof.

Carriers or excipients can also be used to facilitate administration of the GLP-1 receptor agonist compounds. Examples of carriers and excipients include calcium carbonate, calcium phosphate, various sugars such as lactose, glucose, or sucrose, or types of starch, cellulose derivatives, gelatin, vegetable oils, polyethylene glycols and physiologically compatible solvents.

The GLP-1 receptor agonist compounds can also be formulated as pharmaceutically acceptable salts (e.g., acid addition salts) and/or complexes thereof. Pharmaceutically acceptable salts are non-toxic salts at the concentration at which they are administered. Pharmaceutically acceptable salts include acid addition salts such as those containing sulfate, hydrochloride, phosphate, sulfamate, acetate, citrate, lactate, tartrate, methanesulfonate, ethanesulfonate, benzenesulfonate, p-toluenesulfonate, cyclohexylsulfamate and quinate. Pharmaceutically acceptable salts can be obtained from acids such as hydrochloric acid, sulfuric acid, phosphoric acid, sulfamic acid, acetic acid, citric acid, lactic acid, tartaric acid, malonic acid, methanesulfonic acid, ethanesulfonic acid, benzenesulfonic acid, p-toluenesulfonic acid, cyclohexylsulfamic acid, and quiniec acid. Such salts may be prepared by, for example, reacting the free acid or base forms of the product with one or more equivalents of the appropriate base or acid in a solvent or medium in which the salt is insoluble, or in a solvent such as water which is then removed in vacuo or by freeze-drying or by exchanging the ions of an existing salt for another ion on a suitable ion exchange resin.

Exemplary pharmaceutical formulations of GLP-I receptor agonist compounds are described in U.S. Pat. No. 7,521,423, U.S. Pat. No. 7,456,254; US Publication No 2004/0106547, WO 2006/068910, WO 2006/125763, and the like, the disclosures of which are incorporated by reference herein.

The therapeutically effective amount of the GLP-1 receptor agonist compounds described herein for use in the methods described herein will typically be from about 0.01 μg to about 5 mg; about 0.1 μg to about 2.5 mg; about 1 μg to about 1 mg; about 1 μg to about 50 μg; or about 1 μg to about 25 μg. Alternatively, the therapeutically effective amount of the GLP-1 receptor agonist compounds may be from about 0.001 μg to about 100 μg based on the weight of a 70 kg patient; or from about 0.01 μg to about 50 μg based on the weight of a 70 kg patient. These therapeutically effective doses may be administered once/day, twice/day, thrice/day, once/week, biweekly, or once/month, depending on the formulation. The exact dose to be administered is determined, for example, by the formulation, such as an immediate release formulation or an extended release formulation. For transdermal, nasal or oral dosage forms, the dosage may be increased from about 5-fold to about 10-fold.

In other embodiments of the methods described herein, the GLP-1 receptor agonist compound can be administered with (e.g., as separate compositions or in the same composition) an effective amount of an amylin, an amylin analog (e.g., davalintide), GIP, a GIP analog, PYY, a PYY analog, leptin, a leptin analog (e.g., metreleptin), or a combination of two or more thereof. Therapeutically effective amounts of these compounds are known in the art or can be determined by the skilled artisan based on the knowledge in the art and the teachings herein.

### EXAMPLE

The example is for purposes of illustration only and is not intended to limit the scope of the disclosure or claims.

Thirty human patients with type 2 diabetes will take part in a six week, randomized, double-blind, placebo-controlled, parallel group study to determine the impact of exenatide on sleep and circadian function. The study will evaluate, e.g., minutes of NREM sleep; total amount of EEG slow-wave activity; habitual sleep duration and quality; and apnea-hypopnea index (AHI), a validated marker of the severity of obstructive sleep apnea.

Patients will be studied prior to randomization (baseline) and at the end of six weeks of treatment with twice daily subcutaneous injections of BYETTA® (exenatide; Amylin Pharmaceuticals, Inc. and Eli Lilly and Company) (5 μg for the first two weeks, and then 10 μg for the next four weeks) or placebo. An early assessment will be performed after 2 weeks of treatment under both conditions. The baseline study will include assessment of presence and severity of obstructive sleep apnea. The one week home studies will include recordings of habitual sleep duration and quality by wrist actigraphy and sleep logs, in addition to 3 days of meal logs.

Each second inpatient study will involve controlled caloric intake (matching the patient's daily requirements), continuous monitoring of interstitial glucose, two nights of polysomnography, and a 24 hour period of blood sampling at frequent intervals. A fasting blood sample will be drawn for the measurement of CRP, HbAlc, lipids, Apolipoproetin B, and adiponectin levels. The levels of plasma glucose, insulin, C-peptide, free fatty acids, leptin, ghrelin, PYY, melatonin, and catecholamines will be determined on the sample collected during 24 hour blood sampling. Additional measurements will include the 24 hour profile of heart rate variability and blood pressure, and frequent administration of scales to assess hunger, appetite, nausea, and daytime sleepiness. A biopsy of abdominal fat from the peri-umbilical region will be obtained. Tissue samples will be digested collagenase to isolate adipocytes. Insulin sensitivity will be determined by incubating the cells with increasing concentrations of insulin (0-10 nM). Cells will be stimulated for 10 minutes and analyzed by phospho-specific immunoblotting or for 45 minutes pluse 14C-glucose to measure lipogenesis. Alternatively, samples will be cultured in vitro as tissue chunks for analysis of oscillation of circadian gene expression over a 2 day period. Cells will be harvested periodically and gene expression determined by quantitative real time PCR.

The early assessment at 2 weeks after starting treatment will include a one week home study, discussed above, and a one day inpatient study with PSG to assess early effects of exenatide on sleep, CGMS, assessments of hunger, appetite, nausea, daytime sleepiness, 24 hour profile of heart rate variability and blood pressure, fasting blood samples for CRP, lipids, ApoB, and adiponectin.

The primary study endpoints are (i) minutes of non-REM sleep; (ii) total amount of EEG slow-wave activity; (iii) habitual sleep duration and quality; and (iv) AHI, a validated marker of the severity of obstructive sleep apnea. The second study endpoints include (i) glucose control as estimated by mean daytime, nighttime and 24 hour levels and mean peak post-prandial levels of interstitial glucose from 3 days of continuous blood glucose monitoring (CGMS) as well as HAlc; (ii) insulin sensitivity, as estimated by the fasting HOMA index, and insulin signaling directly measured in primary adipocytes using phosphospecific immunoblotting and metabolic assays; (iii) levels of hormones involved in the neuroendocrine control of appetite, including leptin, ghrelin and PYY; (iv) subjective hunger and appetite; (v) phase relationship between melatonin onset (marker of central circadian rhythmicity) and nocturnal peak of leptin profile (marker of peripheral rhythm); and (vi) rhythm of circadian gene expression in primary adipocytes.

All publications, patents, and applications are incorporated by reference herein. The foregoing has been described in detail, and the skilled artisan will recognize that modifications may be made without departing from the spirit or scope of the disclosure or appended claims.

What is claimed is:

1. A method for reducing the symptoms of obstructive sleep apnea in a human in need thereof, the method comprising:
peripherally administering to the human a therapeutically effective amount of a GLP-1 receptor agonist compound, wherein the GLP-1 receptor agonist compound comprises an amino acid sequence having at least 90% sequence identity to GLP-1(7-37) (SEQ ID NO: 22) that is selected from the group consisting of:
GLP-1(7-37) (SEQ ID NO:22);
GLP-1(7-36) (SEQ ID NO:23);
liraglutide;
taspoglutide;
desamino-His⁷,Arg²⁶,Lys³⁴(N^{ε}-(γ-Glu(N-α-hexadecanoyl)))-GLP-1(7-37);
desamino-His⁷,Arg²⁶,Lys³⁴(N^{ε}-octanoyl)-GLP-1(7-37);
Arg^{26,34},Lys³⁸(N^{ε}(ω-carboxypentadecanoyl))-GLP-1(7-38);
Arg³⁴-GLP-1(7-37) (SEQ ID NO:26);
Glu³⁰-GLP-1(7-37) (SEQ ID NO:27);
Lys²²-GLP-1(7-37) (SEQ ID NO:28);
Gly^{8,36},Glu²²-GLP-1(7-37) (SEQ ID NO:29); and
Val⁸,Glu²²,Gly³⁶-GLP-1(7-37) (SEQ ID NO:30).

2. The method of claim 1, wherein the therapeutically effective amount of the GLP-1 receptor agonist compound is 0.01 μg to 5 mg which is administered once per day, twice per day, thrice per day, once per week, biweekly or once per month.

3. The method of claim 1, wherein the therapeutically effective amount of the GLP-1 receptor agonist compound is 0.1 μg to 2.5 mg which is administered once per day, twice per day, thrice per day, once per week, biweekly or once per month.

4. The method of claim 1, wherein the GLP-1 receptor agonist compound is covalently linked to the Fc portion of an immunoglobulin comprising the sequence of SEQ ID NO:41.

5. The method of claim 1, wherein the GLP-1 receptor agonist compound is covalently linked, directly or through a linking group, to one or two polyethylene glycol molecules.

6. The method of claim 1, wherein the GLP-1 receptor agonist compound comprises an amino acid sequence having at least 95% sequence identity to GLP-1(7-37) (SEQ ID NO: 22) that is selected from the group consisting of:
GLP-1(7-37) (SEQ ID NO:22);
GLP-1(7-36) (SEQ ID NO:23);
liraglutide;
Arg³⁴-GLP-1(7-37) (SEQ ID NO:26);
Glu³⁰-GLP-1(7-37) (SEQ ID NO:27); and
Lys²²-GLP-1(7-37) (SEQ ID NO:28).

7. The method of claim 1, wherein the GLP-1 receptor agonist compound comprises an amino acid sequence of liraglutide.
